(19) United States
(12) Patent Application Publication
(10) Pub. No.: US 2025/0239337 A1
(43) Pub. Date: Jul. 24, 2025

(54) INFORMATION MANAGEMENT APPARATUS, OPERATION METHOD OF INFORMATION MANAGEMENT APPARATUS, AND NON-TRANSITORY COMPUTER READABLE MEDIUM

(71) Applicant: FUJIFILM Corporation, Tokyo (JP)

(72) Inventors: Takahiro KANEDA, Tokyo (JP); Tomokazu YAGO, Tokyo (JP)

(73) Assignee: FUJIFILM Corporation, Tokyo (JP)

(21) Appl. No.: 19/032,442

(22) Filed: Jan. 21, 2025

(30) Foreign Application Priority Data
Jan. 22, 2024 (JP) 2024-007166

(51) Int. Cl. G16H 10/60

(52) U.S. Cl. CPC G16H 10/60

(57) ABSTRACT

A pseudo health data generation unit outputs pseudo health data, in which at least any one piece of information is pseudo-generated, in response to an input of request information requested by a user among a plurality of pieces of information. A multifunctional interface unit performs user notification processing of notifying the pseudo health data to the user.

### CROSS-REFERENCE TO RELATED APPLICATIONS

This application claims priority under 35 U.S.C § 119(a) to Japanese Patent Application No. 2024-007166 filed on 22 Jan. 2024. The above application is hereby expressly incorporated by reference, in its entirety, into the present application.

### BACKGROUND OF THE INVENTION

### 1. Field of the Invention

The present invention relates to an information management apparatus, an operation method of an information management apparatus, and a non-transitory computer readable medium storing a computer-executable program that manage health data obtained from a health checkup or the like.

### 2. Description of the Related Art

Health data obtained from a health checkup or the like is used in a pharmaceutical company, a medical university, or the like depending on the research purpose. In a case of using the health data from an examinee who has already taken the health checkup, the user such as a pharmaceutical company needs to obtain use permission from the examinee. In a case of obtaining the use permission, it is necessary to exclude real name information of an individual so that the individual is not specified from the health data as shown in WO2021/085519A (corresponding to US2022/0374550A1).

### SUMMARY OF THE INVENTION

As described above, in a case of using the health data from an examinee who has already taken the health checkup, the user can understand which type of data it is (whether the data indicates that the examinee is healthy, whether the data indicates that the examinee may have a specific disease, or the like) only after obtaining consent of the examinee for the use permission. Therefore, the user needs to request the use permission at random in order to obtain the health data suitable for the request of the user, such as research.

An object of the present invention is to provide an information management apparatus, an operation method of an information management apparatus, and a non-transitory computer readable medium storing a computer-executable program that can provide health data suitable for a request of a user so that it is not necessary to request use permission at random.

An information management apparatus according to an aspect of the present invention comprises a processor, in which the processor outputs pseudo health data, in which at least any one piece of information is pseudo-generated, in response to an input of request information requested by a user among a plurality of pieces of information, and notifies the pseudo health data to the user.

It is preferable that, in the pseudo health data, information other than the request information is pseudo-generated. It is preferable that the processor receives selection of the pseudo health data by the user and extracts actual health data similar to the selected pseudo health data from among a plurality of pieces of the actual health data. It is preferable that the processor notifies a request for use permission to an examinee of the actual health data.

It is preferable that the processor controls presence or absence of extraction of the actual health data with reference to a storage unit that stores the pseudo health data in association with the user. It is preferable that the processor extracts the actual health data in a case where the pseudo health data is selected by the user in the storage unit, and does not extract the actual health data in a case where the pseudo health data is selected by a user who is not associated with the pseudo health data in the storage unit.

It is preferable that, in the processor, the pseudo health data is output from a learning model in a case where the request information is input to the learning model trained using each piece of information. It is preferable that the information includes attribute information including at least any one of blood pressure, age, a blood glucose level, a uric acid level, or a body weight, and an indicator including at least any one of hypertension, metabolic syndrome, or diabetes.

An operation method of an information management apparatus according to an aspect of the present invention comprises: via a processor, a pseudo health data generation step of outputting pseudo health data, in which at least any one piece of information is pseudo-generated, in response to an input of request information requested by a user among a plurality of pieces of information; and a user notification step of notifying the pseudo health data to the user.

A non-transitory computer readable medium for storing a computer-executable program according to an aspect of the present invention causes a processor to execute: a pseudo health data generation function that outputs pseudo health data, in which at least any one piece of information is pseudo-generated, in response to an input of request information requested by a user among a plurality of pieces of information; and a user notification function that notifies the pseudo health data to the user.

According to the present invention, it is possible to provide health data suitable for the request of the user so that it is not necessary to request the use permission at random.

### BRIEF DESCRIPTION OF THE DRAWINGS

FIG. 1 is a schematic diagram of an information management system.

FIG. 2 is a block diagram showing a function of an information management apparatus.

FIG. 3 is an explanatory diagram showing a processing flow of a generation method of pseudo health data.

(A) and (B) of FIG. 4 are explanatory diagrams showing a specific example of a generation method of pseudo health data.

FIG. 5 is an explanatory diagram showing a learning example of a pseudo health data generation unit.

FIG. 6 is an image diagram showing an example of a list display of pseudo health data.

FIG. 7 is an explanatory diagram showing a processing flow of an extraction method of actual health data.

FIG. 8 is an explanatory diagram showing a specific example of an extraction method of actual health data.

FIG. 9 is an explanatory diagram showing a specific example in a case of extracting actual health data.

FIG. 10 is an explanatory diagram showing a specific example in a case where actual health data is not extracted.

FIG. 11 is a flowchart showing a series of flows of a generation method of pseudo health data.

### DESCRIPTION OF THE PREFERRED EMBODIMENTS

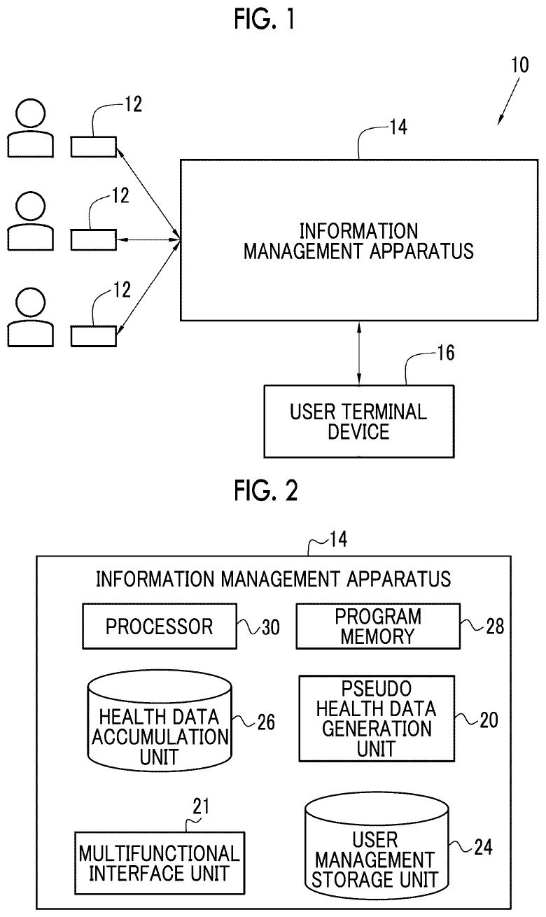

As shown in FIG. 1, an information management system 10 comprises a subject terminal device 12, an information management apparatus 14, and a user terminal device 16. The subject terminal device 12 is used for collecting data obtained from a subject such as an examinee who has taken a health checkup, and transmits or receives various types of data by performing bidirectional notification with the information management apparatus 14. The information management apparatus 14 receives the actual health data output from the subject terminal device 12 and performs various types of processing on the received actual health data. In addition, the information management apparatus 14 is used for utilization of the actual health data in a pharmaceutical company, a university, or the like, and transmits or receives various types of data by performing bidirectional communication with the user terminal device 16.

The actual health data refers to data related to health or medical care of an individual measured by a device owned by a hospital, a health checkup center, an individual, or the like. Actual examination data includes, for example, vital data such as blood pressure and heart rate measured by an individual, life logs such as meals and exercise, health checkup results measured based on a system of a company or a country, and medical records at a hospital.

As shown in FIG. 2, the information management apparatus 14 comprises a pseudo health data generation unit 20, a multifunctional interface unit 21, a user management storage unit 24 (storage unit), and a health data accumulation unit 26. An information management program for performing various types of processing is stored in a program memory 28 in the information management apparatus 14. A processor 30 provided in the information management apparatus 14 reads out and executes the program in the program memory 28 to implement the function of the pseudo health data generation unit 20 (pseudo health data generation function) and the function of the multifunctional interface unit 21 (a plurality of functions including the user notification function). It is preferable that the multifunctional interface unit 21 is configured by an application programming interface (API) server or the like.

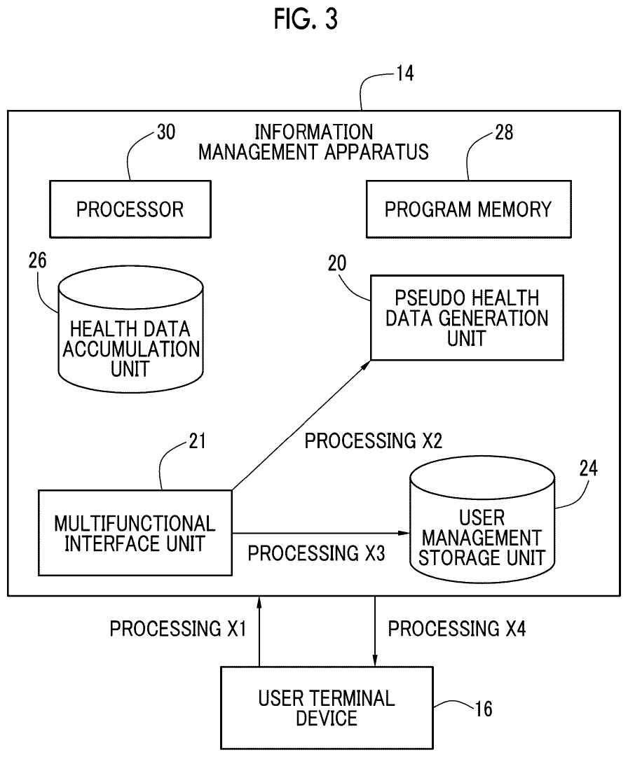

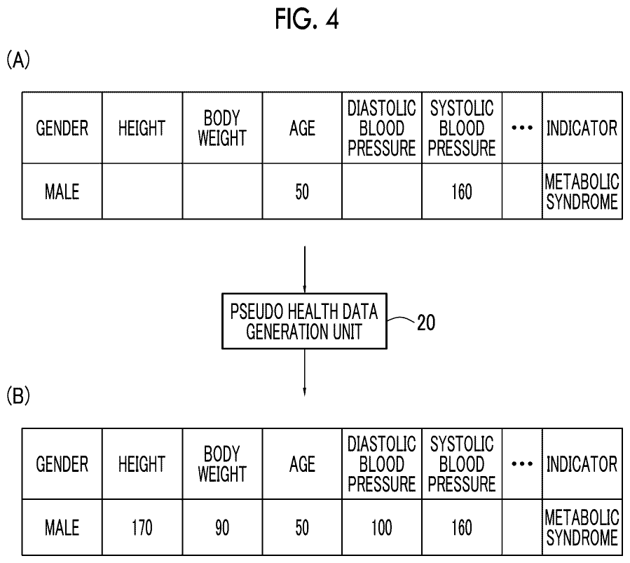

The pseudo health data generation unit 20 outputs pseudo health data, in which at least any one piece of information is pseudo-generated, in response to an input of request information requested by a user among a plurality of pieces of information including the health data. The input of the request information is performed by the user terminal device 16. Specifically, as shown in FIG. 3, in a case where the request information is input from the user terminal device 16 to the information management apparatus 14 (processing X1), the multifunctional interface unit 21 outputs the input request information to the pseudo health data generation unit 20 (processing X2). The pseudo health data generation unit 20 generates the pseudo health data from the request information. The generated pseudo health data is output to the multifunctional interface unit 21.

Then, the multifunctional interface unit 21 performs user notification processing of notifying the pseudo health data to the user who operates the user terminal device 16 (processing X3). In addition, the pseudo health data is stored in the user management storage unit 24 in association with the user (processing X4). In the user management storage unit 24, the pseudo health data associated with the user is stored by using user-related information such as a user ID.

It is preferable that the information included in the actual health data or the pseudo health data includes attribute information including at least any one of blood pressure, age, a blood glucose level, a uric acid level, or a body weight, and an indicator including at least any one of hypertension, metabolic syndrome, or diabetes. It is preferable that the blood pressure includes diastolic blood pressure and systolic blood pressure. For example, in a case where the generation of the pseudo health data is requested in a case of attribute information in which an examinee is male and 50 years old, his systolic blood pressure is 160, and the indicator is metabolic syndrome, among the request information, information corresponding to the request information is described, and the defective health data in which information other than the request information is not described is input to the pseudo health data generation unit 20 as shown in (A) of FIG. 4. The pseudo health data generation unit 20 predicts information other than the request information based on the defective health data. As shown in (B) of FIG. 4, information other than the request information is predicted as 170 for height, 90 for body weight, and 100 for diastolic blood pressure. The predicted information is pseudo-generated by the pseudo health data generation unit 20.

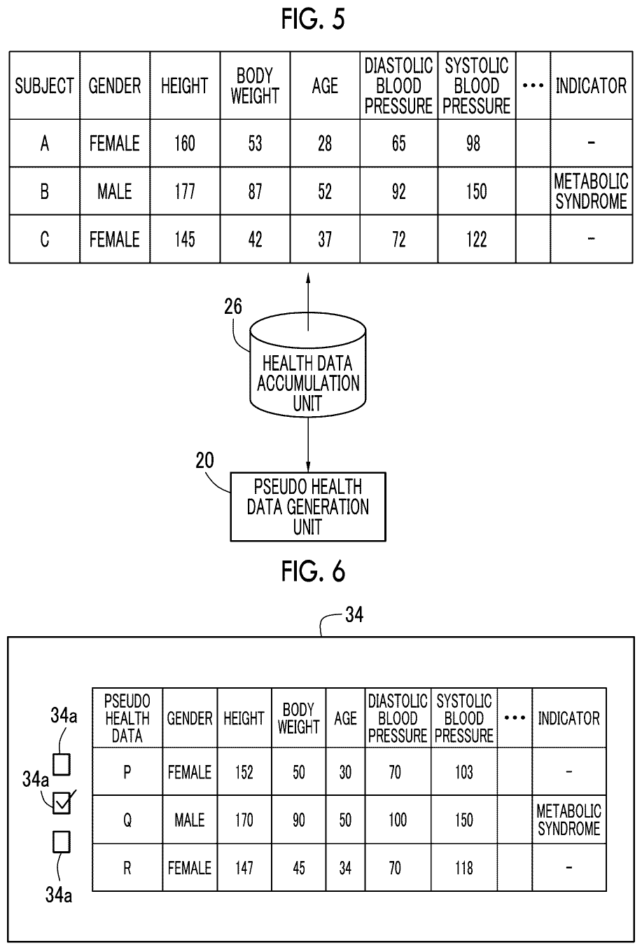

It is preferable that the pseudo health data generation unit 20 is a learning model that outputs the pseudo health data in response to the input of the request information. The learning model is trained using each piece of information. Specifically, as shown in FIG. 5, the pseudo health data generation unit 20 reads out the actual health data from the health data accumulation unit 26 that accumulates a large amount of actual health data including the attribute information. The pseudo health data generation unit 20 performs learning using the read actual health data. In a case of performing learning, it is preferable to perform the learning using health data including both the attribute information and the indicator in addition to the actual health data including the attribute information. For example, in a case where learning is performed using the actual health data of subjects A, B, and C who take the health checkup, the actual health data of the subjects A and C is only the attribute information, but the actual health data of the subject B includes an indicator analyzed as “metabolic syndrome” in addition to the attribute information. The health data accumulation unit 26 may be provided inside the information management apparatus 14, or may be provided outside the information management apparatus 14 in a state of being able to communicate with the information management apparatus 14.

In the user terminal device 16, in a case where the pseudo health data is received, the received pseudo health data is displayed on a display. Specifically, as shown in FIG. 6, a list of the received pseudo health data is displayed on a display 34. In addition, each pseudo health data is provided with a check box 34a so that the user can select each pseudo health data. The user performs operation of checking the check box 34a to select the pseudo health data. In FIG. 6, pseudo health data Q is selected among the pseudo health data P, Q, and R. Information regarding the selection of the pseudo health data is transmitted to the information management apparatus 14.

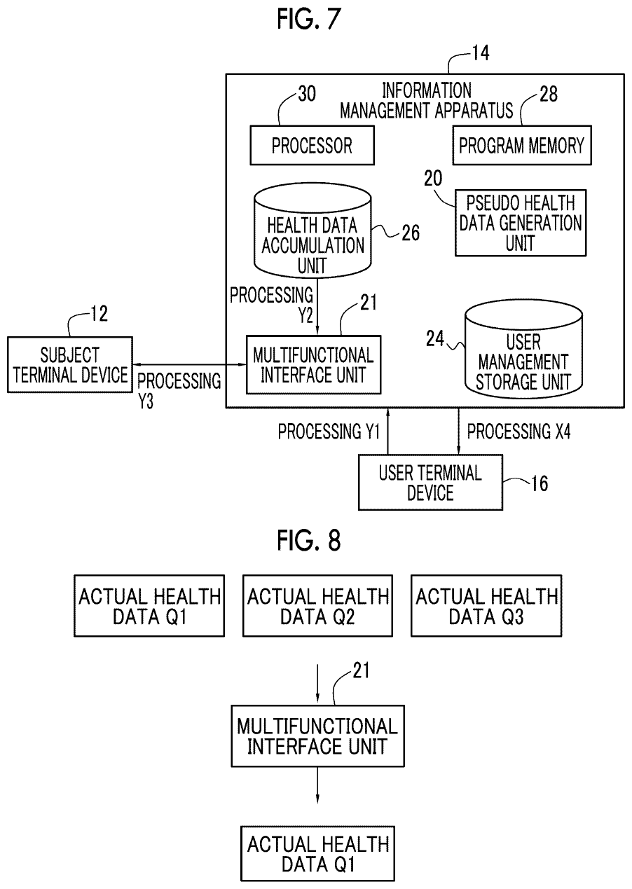

As shown in FIG. 7, the multifunctional interface unit 21 performs reception processing of receiving the selection of the pseudo health data from the user terminal device 16 (processing Y1). In a case where the reception processing is performed, the multifunctional interface unit 21 performs extraction processing of extracting the actual health data similar to the selected pseudo health data from among the plurality of pieces of actual health data with reference to the health data accumulation unit 26 (processing Y2). In a case where the extraction processing is performed, permission request notification processing of notifying a request for use permission to an examinee of the extracted actual health data is performed (processing Y3). The subject terminal device 12 is used for the permission request notification processing. Then, the multifunctional interface unit 21 outputs the actual health data for which the use permission is obtained to the user terminal device 16 (processing Y4).

Specifically, as shown in FIG. 8, in a case where the pseudo health data Q is selected by the user terminal device 16, the multifunctional interface unit 21 extracts actual health data Q1, Q2, and Q3 similar to pseudo health data B. The actual health data Q1 for which the use permission is obtained among the actual health data Q1, Q2, and Q3 is output to the user terminal device 16.

The multifunctional interface unit 21 may control whether or not the actual health data is extracted from the health data accumulation unit 26 with reference to the user management storage unit 24. Specifically, in a case where the received pseudo health data is selected by the user who is associated with the received pseudo health data in the user management storage unit 24, the actual health data is extracted from the health data accumulation unit 26. On the other hand, in a case where the received pseudo health data is selected by the user who is not associated with the received pseudo health data in the user management storage unit 24, the actual health data is not extracted from the health data accumulation unit 26. The user management storage unit 24 may be provided inside the information management apparatus 14, or may be provided outside the information management apparatus 14 in a state of being able to communicate with the information management apparatus 14.

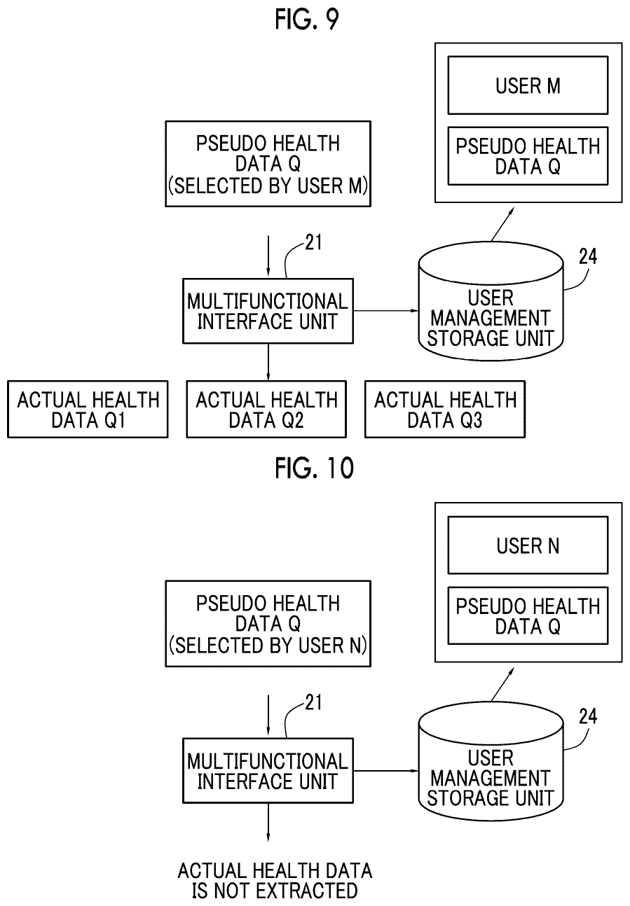

For example, as shown in FIG. 9, in a case where the pseudo health data Q associated with a user M is stored in the user management storage unit 24, the actual health data Q1, Q2, and Q3 similar to the pseudo health data Q are extracted in a case where the user M selects the pseudo health data Q. In this case, it is preferable to display a message indicating that extraction is possible on the user terminal device 16. On the other hand, as shown in FIG. 10, in a case where the pseudo health data B is selected by a user N who is not associated with the pseudo health data B, the actual health data Q1, Q2, and Q3 similar to the pseudo health data Q are not extracted. In this case, it is preferable to display a message indicating that extraction is not possible on the user terminal device 16.

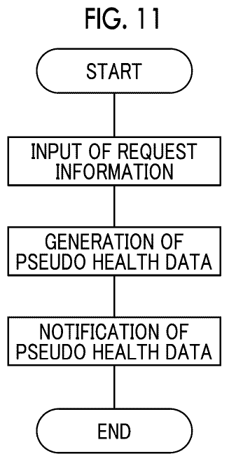

Next, a series of flows of a generation method of the pseudo health data will be described with reference to a flowchart shown in FIG. 11. The user inputs the request information requested by the user with the user terminal device 16. The input request information is output to the pseudo health data generation unit 20 via the multifunctional interface unit 21. Accordingly, the pseudo health data generation unit 20 outputs the pseudo health data, in which at least any one piece of information is pseudo-generated, in response to the input of the request information requested by the user among the plurality pieces of information including the health data (pseudo health data output step). The output pseudo health data is notified to the user via the multifunctional interface unit 21 and the user terminal device 16 (user notification step).

In the above-described embodiment, the hardware structures of processing units that execute various types of processing, such as the pseudo health data generation unit 20 and the multifunctional interface unit 21, are various processors as described below. The various processors include a central processing unit (CPU) that is a general-purpose processor that executes software (programs) to function as various processing units, a graphics processing unit (GPU), a programmable logic device (PLD) that is a processor capable of changing a circuit configuration after manufacture, such as a field programmable gate array (FPGA), and an exclusive electric circuit that is a processor having a circuit configuration exclusively designed to execute various types of processing.

One processing unit may be composed of one of the various processors or may be composed of a combination of two or more processors of the same type or different types (for example, a combination of a plurality of FPGAs, a combination of a CPU and an FPGA, or a combination of a CPU and a GPU). In addition, one processor may constitute a plurality of processing units. As an example in which one processor constitutes a plurality of processing units, first, there is an aspect where one processor is formed of a combination of one or more CPUs and software and the processor functions as the plurality of processing units, as represented by a computer such as a client and a server. Second, there is an aspect where a processor, which implements functions of the entire system including the plurality of processing units with one integrated circuit (IC) chip, is used, as represented by a system on a chip (SoC), or the like. As described above, various processing units are composed of one or more of the above-described various processors as hardware structures.

Further, the hardware structures of these various processors are, more specifically, electric circuits (circuitry) in a form in which circuit elements such as semiconductor elements are combined. In addition, a hardware structure of the storage unit is a storage device such as a hard disc drive (HDD) and a solid state drive (SSD).

### EXPLANATION OF REFERENCES

10: information management system
12: subject terminal device
14: information management apparatus
16: user terminal device
20: pseudo health data generation unit
21: multifunctional interface unit
24: user management storage unit (storage unit)
26: health data accumulation unit
28: program memory
30: processor
34: display
34a: check box

What is claimed is:

1. An information management apparatus comprising:
a processor,
wherein the processor is configured to:
output pseudo health data, in which at least one piece of information is pseudo-generated, in response to an input of request information requested by a user among a plurality of pieces of information; and
notify the pseudo health data to the user.

2. The information management apparatus according to claim 1,
wherein in the pseudo health data, information other than the request information is pseudo-generated.

3. The information management apparatus according to claim 1,
wherein the processor is configured to:
receive selection of the pseudo health data by the user; and
extract actual health data that is similar to the selected pseudo health data from among a plurality of pieces of the actual health data.

4. The information management apparatus according to claim 3,
wherein the processor is configured to notify a request for use permission to an examinee of the actual health data.

5. The information management apparatus according to claim 3,
wherein the processor is configured to control whether the actual health data is extracted by referring to a storage unit that stores the pseudo health data in association with the user.

6. The information management apparatus according to claim 5,
wherein the processor is configured to:
extract the actual health data in a case where the pseudo health data is selected by the user in the storage unit; and
does not extract the actual health data in a case where the pseudo health data is selected by a user who is not associated with the pseudo health data in the storage unit.

7. The information management apparatus according to claim 5,
wherein the processor is configured to notify a request for use permission to an examinee of the actual health data in a case where the actual health data is extracted.

8. The information management apparatus according to claim 1,
wherein the processor is configured to output the pseudo health data from a learning model in a case where the request information is input to the learning model that is trained using each piece of information.

9. The information management apparatus according to claim 1,
wherein the information includes attribute information including at least one of blood pressure, age, blood glucose level, uric acid level, or body weight, and also includes an indicator containing at least one of hypertension, metabolic syndrome, or diabetes.

10. An operation method of an information management apparatus comprising:
via a processor,
a pseudo health data generation step of outputting pseudo health data, in which at least one piece of information is pseudo-generated, in response to an input of request information requested by a user among a plurality of pieces of information; and
a user notification step of notifying the pseudo health data to the user.

11. A non-transitory computer readable medium for storing a computer-executable program, the computer-executable program causing a processor to execute:
a pseudo health data generation function that outputs pseudo health data, in which at least one piece of information is pseudo-generated, in response to an input of request information requested by a user among a plurality of pieces of information; and
a user notification function that notifies the pseudo health data to the user.
